(19) United States
(12) Patent Application Publication
(10) Pub. No.: US 2024/0298593 A1
(43) Pub. Date: Sep. 12, 2024

(54) WATER EFFICIENT CUCURBITACEAE

(71) Applicant: ORIGENE SEEDS LTD, Givat Brenner (IL)

(72) Inventors: Hamutal POMPAN, Givat Brenner (IL); Eyal VARDI, Givat Brenner (IL)

(21) Appl. No.: 18/268,324

(22) PCT Filed: Dec. 1, 2021
(86) PCT No.: PCT/IL2021/051429

§ 371 Date: Jun. 20, 2023

Related U.S. Application Data
(60) Provisional application No. 63/133,792, filed on Jan. 5, 2021.

(51) Int. Cl. A01H 6/34; A01H 1/00; A01H 5/08

(52) U.S. Cl. CPC A01H 6/342; A01H 1/1225; A01H 5/08

(57) ABSTRACT

The present invention is in the field of agriculture and in particular in the field of solutions to severe agriculture environments for providing a solution to drought conditions, also in vegetable crops like watermelon.

More specifically, the present invention includes developing and cultivating watermelon hybrids producing beneficial low quantities of water to high fruit yield thus providing watermelon crops as a desirable fruit for human consumption tolerant to water deficits and environmental factors characterized by a genetic improvement of stress tolerance in plants and a tolerance to water feed conditions of less than 190-210 M3 per 1000 m2.

### TECHNOLOGICAL FIELD

The present disclosure is in the field of agriculture and in particular in the field of solutions to severe agriculture environments.

### BACKGROUND ART

References considered to be relevant as background to the presently disclosed subject matter are listed below:
Akashi K, Morikawa K, Yokota A, 2005. Agrobacterium-mediated transformation system for the drought and excess light stress-tolerant wild watermelon (Citrullus lanatus). Plant Biotech. 22(1): 13-18.
Bertucci M B, Suchoff D H, Jennings K M, Monks D W, Gunter C C, Schultheis J R, Louws F J, 2018. Comparison of Root System Morphology of Cucurbit Rootstocks for Use in Watermelon Grafting. HortTechnology 28 (5): 629-636.
Boyer J S, 1982. Plant productivity and environment. Sci. 218 (4571):443-8.
Freeman, S, 2014. Biological Sciences. United States of America: Pearson. pp. 765-766. ISBN 978-0-321-74367-1.
Fricker & Willmer, C, 1996. Stomata. Springer Netherlands. p. 18. ISBN 978-94-011-0579-8. Retrieved 15 Jun. 2016.
Graham, L E, 2006. Plant Biology. Upper Saddle River, NJ 07458.
Hill J O, Simpson R J, Moore A D, Chapman D F, 2006. Morphology and response of roots of pasture species to phosphorus and nitrogen nutrition. Plant & Soil 286 (7).
Kumar P, Rouphael Y, Cardarelli M, Colla G, 2017. Vegetable grafting as a tool to improve drought resistance and water use efficiency. Front Plant Sci. 8:1130.
Park M, Won H, Kim J, Han J, Ahn Y, 2012. Expression of H+ phosphatase from Arabidopsis enhances the resistance of drought stress in transgenic bottle gourd rootstock (Lagenaria siceraria Standl.). Cucurbitaceae 2012.
Pearson Education, Inc. pp. 200-202. ISBN 978-0-13-146906-8.
Pritchard, J, 2001. “Turgor Pressure”. Encyclopedia of Life Sciences. American Cancer Society. ISBN 9780470015902.
Poor R E, 2015. Investigating the effect of grafted watermelon on tolerance to drought and salinity. JANS J. 4-6:670-673.
Proietti S, Rouphael Y, Colla G, Cadarelli M, De Agazio M, Zacchii M, Rea E, Moscatello S, Battistelli A, 2008. Fruit quality of mini-watermelon as affected by grafting and irrigation regimes. J Sci of Food & Ag. 88(6): 1107-1114.
Sinha, R K, 2004. Modem Plant Physiology. CRC Press. ISBN 978-0-8493-1714-9.
Steudle, E, 1977. Plant Physiology. 59 (2): 285-9.
Taiz, L, 2015. Plant Physiology and Development. Sunderland, MA: Sinauer Associates, Inc. p. 101. ISBN 978-1-60535-255-8.
Tetteh A Y, Whener T C, Davis A R, 2010. Identifying resistance to powdery mildew race 2W in the USDA-ARS watermelon germplasm collection. Crop Sci. 50: 933-393.
Yanling M, Ruiping Y, Lianhong L, Xiurong G, Xiaozhen Y, Yongqi W, Xian Z, Hao L. 2015. Growth, photosynthesis and adaptive responses of wild and domesticated watermelon genotypes to drought stress and subsequent re-watering. Plant Growth Regul. 79(2): 229-241.
Waizel Y, Eshel A, Kafkafi U, 1996. Plant Roots: The Hidden Half. Second Edition. Marcel Dekker Inc., New-York.
Yasar et al., 2012. Accumulation and distribution of iron, zinc and manganese ions in pumpkin (Cucurbita spp.) and gourd (Lagenaria siceraria) accessions subjected to drought stress. Cucurbitaceae.
Zhang H, Gong G, Gou S, Ren Y, Xu Y, 2011. Screening the USDA Watermelon Germplasm Collection for Drought Tolerance at the Seedling Stage. HortSci. 46(9): 1245-1248.

Acknowledgement of the above references herein is not to be inferred as meaning that these are in any way relevant to the patentability of the presently disclosed subject matter.

### FIELD AND BACKGROUND

Watermelon (Citrullus lanatus (Thunb.) Matsum. And Nakai), family Cucurbitaceae, is one of the most economically important and widely cultivated vegetable crops in many areas worldwide and includes both seeded and seedless fruit. During the last 50 years, the world production area of watermelons has increased by 62%, from 1.96 million ha in 1961 to 3.16 million ha in 2012. During the same period, the yield tripled, resulting in a fivefold total production increase (from 17.8 to 89.0 million tons).

The top 10 world watermelon producers include China (accounting for 63% of the production in 2010), followed by Turkey, Iran, Brazil, USA, Egypt, Uzbekistan, Russia, Mexico and Algeria (FAOSTAT 2012 in Jensen, 2012, Cucurbitaceae 2012 p. 264-273).

In the United States, watermelons are considered a major vegetable crop. Farmers in 44 States grow more than $500 million worth of watermelon commercially every year (USDA, 2017). The main production states are Florida, California, Arizona, Texas and Georgia. In 2005, the total production of watermelons in the United States was 1.7 million Kg, with a farm value of 410 million US$ (USDA, 2006 IN Tetteh et al., 2010, Crop Sci. 50:933-939).
In Turkey, watermen production in 2012 was 4 million tons (165,000 hectare).
In Iran, watermen production in 2012 was 3.8 million tons (145,000 hectare).
In Brazil watermen production in 2012 was 2 million tons (95,000 hectare).

In this arena, drought is considered a major abiotic stress factor affecting watermelon productivity (Boyer, 1982, Zhang et al., 2011) and has progressed to alarming levels worldwide, including USA, South America countries, the Middle East countries, Russia and Far East countries. Therefore, the genetic improvement of stress tolerance in plants is an urgent challenge for the future of agriculture (Akashi et al., 2005).

In certain regions worldwide, water supplies are scarce and water management is a necessity. Water cost is very high in areas where water availability is a serious problem for agriculture management.

It is well known in the art that non-grafted watermelons are irrigated with approximately 700-900 liters per plant and grafted watermelons are irrigated with 1400-1800 liters per plant.

For example, In Israel the cost of irrigate 2,500 hectares of grafted watermelon production is approximately 231,437,500 NIS (2,500 hectaresx2.5 NIS/M³×3750 M³/hectare). In USA the water consumption for watermelons is at least 168,750,000 M³ of water (45,000 hectaresx3750 M³/hectare) as the majority of watermelons in the USA are non-grafted.

As appreciated by those versed in the art, there is a long-lasting need to provide a solution to drought conditions. Zhang et al., 2011 describe plants in the early germination stage but provides no motivation or solution for watermelons transplanting after nursery, through all the growing season till harvest of the mature fruit. Yasar et al., 2012 describe PEG for simulation of drought conditions and Park et al. 2012 describe theoretical transgenic plants resistant to drought stress.

Vegetable grafting using rootstocks has emerged as a rapid tool in tailoring plants to suboptimal growth conditions (Kumar et al., 2017; Poor, 2015; Proietti et al., 2008). While there is a lot of information on grafting improving the tolerance of watermelon to drought conditions, there is a need for understanding the adaptive responses of wild and domesticated watermelon genotypes to drought stress (Yanling et al., 2015).

Watermelon (Citrullus lanatus) is a plant species in the family Cucurbitaceae, a vine-like flowering plant originally domesticated in West Africa. It is a highly cultivated fruit worldwide, having more than 1,000 varieties.

Watermelon is a scrambling and trailing vine in the flowering plant family Cucurbitaceae. There is evidence from seeds in Pharaoh Tombs of watermelon cultivation in Ancient Egypt. Watermelon is grown in favorable climates from tropical to temperate regions worldwide for its large edible fruit, which is a berry with a hard rind and no internal divisions and is botanically called a pepo. The sweet, juicy flesh is usually deep red to pink, with many black seeds, although seedless varieties exist. The fruit can be eaten raw or pickled, and the rind is edible after cooking. It is commonly consumed as a juice or as an ingredient in mixed beverages.

Considerable breeding effort has developed disease-resistant varieties. Many cultivars are available that produce mature fruit within 85-100 days of planting. In 2017, China produced about two-thirds of the world total of watermelons.

Evidence of the cultivation of both Citrullus lanatus and Citrullus colocynthis in the Nile Valley has been found from the second millennium BC onward, and seeds of both species have been found at Twelfth Dynasty sites and in the tomb of Pharaoh Tutankhamun. Watermelon seeds were found in the Dead Sea region at the ancient settlements of Bab edh-Dhra and Tel Arad.

In the 7th century, watermelons were being cultivated in India, and by the 10th century had reached China, which is today the world's single largest watermelon producer. The Moors introduced the fruit into the Iberian Peninsula and there is evidence of it being cultivated in Córdoba in 961 and in Seville in 1158. It spread northwards through southern Europe, perhaps limited in its advance by summer temperatures being insufficient for good yields. The fruit had begun appearing in European herbals by 1600 and was widely planted in Europe in the 17th century as a minor garden crop.

According to the teachings known in the art, Healthy watermelon vines produce 2-4 fruits per plant. The vines produce both male and female flowers. Both are needed to set fruit and there are fewer female flowers compared to male, about one female for every seven males.

Part of wild, unselected forms of watermelon tend to bear bitter fruit, due to the presence of cucurbitacin (a biochemical compound used for defense against herbivores in the wild) and hence are normally only fed to cattle.

Characteristics of the Species Citrullus lanatus are known in the art to include being recognizable by its large fruit which is unique in the Cucurbitaceae of southern Africa and also by the dense yellowish to brownish hairs on the younger plant parts.

The Citrullus lanatus watermelon is an annual plant with long, weak, trailing or climbing stems which are five-angled and up to 3 m (10 ft.) long. The young parts are densely woolly with yellowish to brownish hairs while the older parts become hairless.

The tendrils are rather robust and usually divided in the upper part. Leaf stalks (petioles) up to about 19 cm long, somewhat hairy. The leaves usually have three lobes which are themselves further divided into lobed or doubly lobed. Leaf blades up to about 20×20 cm, more or less hairy, usually deeply 3-5 lobed, the central lobe being the largest.

The leaves are herbaceous but rigid, becoming rough on both sides; 60-200 mm long and 40-150 mm broad, ovate in outline, sometimes unlobed and entire, but usually deeply 3-lobed with the segments again lobed or doubly lobed; the central lobe is much the largest. The leaf stalks are somewhat hairy and up to 150 mm long.

The flowers grow singly in the leaf axils and the corolla is white or yellow inside and greenish-yellow on the outside. The flowers selected are unisexual, with male and female flowers occurring on the same plant (monoecious).

The receptacle ovule is up to about 4 mm long, broadly campanulate and hairy, the lobes are as long as the tube.

The corolla is usually green or green-veined outside and white to pale or bright yellow inside and up to 30 mm in diameter.

Both male and female flowers are yellow, up to 3-4 cm in diameter, and borne on pedicels (flower stalks) up to 40-45 mm long and hairy. The male flowers predominate at the beginning of the season and the female flowers, which develop later, have inferior ovaries (carpel). The styles are united into a single column and the large fruit is a kind of modified berry called a pepo. This has a thick rind (exocarp) and fleshy center (mesocarp and endocarp).

Vines produce male and female flowers separately on the same plant. They often begin producing male flowers several days before the females appear. Commonly, male flowers fall off and female flowers will stay on the vine and bear fruit.

The fruits of wild plants are sub globose, indehiscent (such as, by way of non-limiting examples only, Citrullus colocynthis or Citrullus citroides); and the fruit stalk is up to 50 mm long, greenish mottled with darker green.

In the wild forms (genotypes) the rind is pale or grey-green, usually mottled with irregular longitudinal bands of dark green or grey-green. The flesh in the wild form is firm and rather hard, white, green-white or yellowish.

Fruits of cultivated plants are up to about 70×30 cm, rounded, oval or oblong, with a golden-yellow to dark green rind, the rind being uniform, mottled, or striped. Flesh is usually red or yellow, sometimes orange, pink or white.

In cultivated forms, the rind is often concolorous yellowish to pale or dark green, or mottled with darker green, or including stripes of pale or dark green or marbled with a darker shade. The flesh is somewhat spongy in texture but very juicy and “soft” with varying degrees of firmness, pink to bright red-pink and even dark red but invariably softer than the rind.

There are more than 1,000 cultivars of watermelon range in weight from less than one to more than 90 kilograms (200 lbs.), The flesh of them can be red, orange, yellow or white.

The seeds are numerous, ovate in outline, sometimes bordered; in wild forms they are usually black or dark brown; in cultivated forms they are also white or mottled, mostly 6-12 mm long. Watermelons are grown from seed.

Commonly, Citrullus lanatus grows in grassland and bushland, mostly in sandy soils, often along watercourses or near water. It has been collected at altitudes of 0-1785 m. In southern Africa the flowering time of Citrullus lanatus is mostly from January to April and the fruiting time mostly from February to May. Dry or rainy years will influence flowering and fruiting.

Invariably, at least three months of reliably hot, sunny weather are required to grow and ripen a watermelon. During that time your average daily, maximum temperature should be at least about 20-25° C. or 70-80 F. Warmer is even better.

It is well known in the art that growing watermelons in full sun and harsher climates requires an abundant supply of water and nutrients (good soil). Furthermore, loamy, well-drained soil with a soil pH between 6 and 6.8 are advantageous to Citrullus lanatus crops.

Watermelons grow male and female flowers on the same vine. The smaller male flowers appear first whilst the female flowers are much larger.

Absence of prevalent large female flowers may be indicative of climate extremes of being too hot, too cold, insufficient water or insufficient nutrients. Furthermore, it is well known in the art that occasioning on the Citrullus lanatus plant producing female flowers but the little fruit at the base of the Citrullus lanatus plant “shriveling”, a probable cause thereto may be that the flowers are not getting pollinated.

Invariably, watermelon flowers are insect pollinated. Several methods of human assisted pollination are known in the art, usually performed early in the morning by pulling off a few male flowers and removing the flower petals.

Thereafter, brushing the pollen laden stamen against the stigma in the center of the female flower, so the pollen sticks to thereto.

Some growers practice pollinating the first few female flowers on each branch for the purpose of producing better fruit by of growing the fruit as large as possible and by way of pinching out the tip of the branch after a couple of fruits have set (starting to swell up).

In a 100 gram serving, watermelon fruit provides (on average) around 30 calories and low amounts of essential nutrients. Only vitamin C is present in appreciable content at 10% of the Recommended Daily Allowance (RDA). Watermelon fruit includes, on average, 91% water, 6% sugars, and is low in fat (see table below).

TABLE 1
Nutritional value per 100 g (3.5 oz.) of Watermelon:

| Energy | 127 kJ | (30 kcal) |
|---|---|---|
| Carbohydrates | 7.55 | g |
| Sugars | 6.2 | g |
| Dietary fiber | 0.4 | g |
| Fat | 0.15 | g |
| Protein | 0.61 | g |
| Vitamin A equiv. | 28 μg | (4%) |
| Beta-carotene | 303 μg | (3%) |
| Thiamine (B1) | 0.033 mg | (3%) |
| Riboflavin (B2) | 0.021 mg | (2%) |
| Niacin (B3) | 0.178 mg | (1%) |
| Pantothenic acid (B5) | 0.221 mg | (4%) |
| Vitamin B6 | 0.045 mg | (3%) |
| Choline | 4.1 mg | (1%) |
| Vitamin C | 8.1 mg | (10%) |
| Calcium | 7 mg | (1%) |
| Iron | 0.24 mg | (2%) |
| Magnesium | 10 mg | (3%) |
| Manganese | 0.038 mg | (2%) |
| Phosphorus | 11 mg | (2%) |
| Potassium | 112 mg | (2%) |
| Sodium | 1 mg | (0%) |
| Zinc | 0.1 mg | (1%) |
| Water | 91.45 | g |
| Lycopene | 4532 | μg |

Units: μg = micrograms; mg = milligrams* IU = International units.
Percentages are roughly approximated using US recommendations for adults.
Source: USDA Nutrient Database

Water cost and scarcity is very high in areas where water availability is a serious problem for agriculture management. Therefore, the present disclosure is based on the unique development of hybrid watermelon plants with agriculturally acceptable tolerance to drought conditions and capable of producing edible fruits. Such hybrids have been determined to be a solution for watermelon cultivation in severe agriculture environments, where drought prevents growing edible watermelon suitable for human consumption.

Charles Fredric Andrus, a horticulturist at the USDA Vegetable Breeding Laboratory in Charleston, South Carolina, set out to produce a disease-resistant and wilt-resistant watermelon. The result, in 1954, was “that gray melon from Charleston”. Its oblong shape and hard rind made it easy to stack and ship. Its adaptability meant it could be grown over a wide geographical area. It produced high yields and was resistant to the most serious watermelon diseases: anthracnose and fusarium wilt. Others were also working on disease-resistant varieties; J. M. Crall at the University of Florida produced “Jubilee” in 1963 and C. V. Hall of Kansas State University produced “Crimson sweet” the following year. These are no longer grown to any great extent, but their lineage has been further developed into hybrid varieties with higher yields, better flesh quality and attractive appearance. Another objective of plant breeders has been the elimination of the seeds which occur scattered throughout the flesh. This has been achieved using triploid varieties, but these are sterile, and the cost of producing the seed, through crossing a tetraploid parent with a normal diploid parent, is high.

Today, farmers in approximately 44 states in the United States grow watermelon commercially. Georgia, Florida, Texas, California, and Arizona are the United States' largest watermelon producers. These popular fruit is often large enough that groceries often sell half or quarter melons. Some smaller, spherical varieties of watermelon, both red and yellow-fleshed, are sometimes called “icebox melons” or mini watermelons. The largest recorded watermelon fruit was grown in Tennessee in 2013 and weighed 159 kilograms (351 pounds).

Citrullus lanatus fruits come in sizes ranging from 1.3 kg to 32 kg (3 pounds to over 70 pounds), with either red or yellow/orange flesh. Jubilee, Charleston Grey and Congo are large, cylindrical varieties, while Sugar Baby and Ice Box are two smaller, globe shaped types.

According to the teachings known in the art, Citrullus lanatus seeds need to germinate at a temperature over 70 degrees F. (21 Celsius) In colder climates, planting the seeds directly in the ground is recommended after the last frost, when the temperature is holding steady at above 70 degrees F. (21 Celsius) is recommended.

Prior to planting, covering soil with black plastic to hasten soil warming is often used. Because watermelons are heavy feeders, preparing the planting bed by adding seaweed, compost, or fertilizer is commonly performed for enhanced results. For best nutrient uptake, the soil pH should be between 6 and 6.8, although the plants will tolerate a pH as low as 5. Another option known in the art is to excavate the soil 1 foot deep (33 cm), add a 9-inch-thick (23 cm) layer of manure or fertilizer, and then covering with 3 inches (7.5 cm) of soil mixed with compost, thus creating a bed with a high-nitrogen soil base that is naturally warm. Some gardeners even plant melons in their compost piles to ensure a warm footing and adequate nitrogen.

Commonly, Citrullus lanatus require planting with spacing plants 3 to 5 feet apart from each other. After planting, covering seedlings with floating row covers is commonly performed to keep out insects and trap warm air near plants.

Commonly, mulching with black plastic serves multiple purposes including: trapping moisture, warming the soil, hindering weed growth, and keeping developing fruits\clean.

Due to the difficulty in move among vines at a later stage without crushing them, tackling weeds before vines start to run is commonly performed.

Water plays an important role in keeping vines healthy and producing better fruit. Vines are most sensitive to drought between planting to the time fruits start to form. Avoiding overhead watering, soaker hoses or drip irrigation deliver water directly to soil, helping prevent possible spread of fungal diseases among wet foliage.

Keeping soil consistently moist, but not waterlogged, is beneficial to the reduction of plant fatality. It is typical for leaves to wilt under midday sun, nevertheless, they should not remain wilted into evening. Watering vines early in the morning so leaves can dry before sunset, further helps preventing fungal diseases.

Keeping soil moist but not waterlogged is a further important factor. Watering at the vine's base in the morning conjunctively with trying to avoid wetting the leaves and avoiding overhead watering as well as reducing watering once fruit are growing together with dry weather are considered the best parameters for producing the sweetest melon.

Many farmers choose to fertilize whilst ascertaining delivering more nitrogen than phosphorus and potassium. However, after flowering begins, many farmers use a fertilizer with less nitrogen or using liquid seaweed.

Some farmers like to switch fertilizer during the growing season by using a fertilizer with more nitrogen than phosphorus and potassium during the period between planting and when the first flowers open. Once flowering begins, farmers use a fertilizer with less nitrogen and more phosphorus and potassium, such as African violet food or liquid seaweed.

In colder regions, removal of any blossoms that start to develop within 50 days of the area's first average frost date is common for the purpose of ensure that remaining, larger fruits will ripen before frost.

As fruit is ripening, preventing rotting by gently lifting the fruit and placing some cardboard or straw between the fruit and the soil, can be practiced.

Furthermore, farmers often keep ripening watermelon fruit from direct contact with soil to prevent rot and protect fruit from pests and rodents. Setting fruit on a light-reflective surface, such as aluminum foil, will concentrate heat and speed up ripening. To protect ripening watermelons from large rodents, such as groundhogs, farmers can use cover them with laundry baskets weighted down with a few bricks.

Major pests of the watermelon include aphids, fruit flies and root-knot nematodes, on top of that viruses, bacteria and fungi. In conditions of high humidity, the plants are prone to plant diseases such as powdery mildew and mosaic virus. Some varieties often grown in Japan and other parts of the Far East are susceptible to fusarium wilt. Grafting such varieties onto disease-resistant rootstocks offers protection.

The US Department of Agriculture recommends using at least one beehive per acre (one hive per 4,000 m2 or 0.4 hectares per hive) for pollination of conventional, seeded varieties for commercial plantings. On the other hand, seedless hybrids have sterile pollen. This requires planting pollinizer rows of varieties with viable pollen. Since the supply of viable pollen is reduced and pollination is much more critical in producing the seedless variety, the recommended number of hives per acre (0.4 hectares) (pollinator density) increases to three hives per acre (1,300 m2 or 0.13 hectares per hive). Lack of pollen is thought to contribute to “hollow heart” which causes the flesh of the watermelon to develop a large hole, sometimes in an intricate, symmetric shape. Nevertheless, watermelons suffering from hollow heart are safe to consume.

Farmers of the Zentsuji region of Japan found a way to grow cubic watermelons by growing the fruits in metal and glass boxes and making them assume the shape of the receptacle. The cubic shape was originally designed to make the melons easier to stack and store, but these “square watermelons” may be triple the price of normal ones, so appeal mainly to wealthy urban consumers. Pyramid-shaped watermelons have also been developed and any polyhedral shape may potentially be used. Invariably, cubic shape and pyramid-shaped watermelons suffer from inconsistent levels of ripeness and hence are used mainly for ornamental purposes.

Therefore, there is a long-lasting need to provide a solution to drought conditions, also in vegetable crops like watermelon.

Thus, it would be highly desirable to develop and cultivate watermelon hybrids producing beneficial low quantities of water to high fruit yield thus providing watermelon crops as a desirable fruit for human consumption.

Furthermore, it would be highly desirable to develop and cultivate a watermelon plant tolerant to water deficits and environmental factors characterized by a genetic improvement of (abiotic and/or biotic) stress tolerance in plants and a tolerance to water feed conditions of less than 190-210 M3 per 1000 m2 and having properties such that seedless fruits are produced with a flesh firmness between 2.5-3.5 lbs, measured by PENETROMETER FRUIT PRESSURE TESTER mod. FT 011 (0-11 lbs.), IRC using an 11 mm plunger attachment.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a top view of a field with a known-commercial control variety (Fascination) control group according to preferred embodiments of the present invention obtained by selective backcrossing of carefully selected and matched varieties of Citrullus lanatus according to the present invention.

FIGS. 2A-2B are images of leaf of drought tolerant plant (FIG. 2A) vs. leaves of sensitive plant (FIG. 2B) grown in a net house under conditions of continues drought.

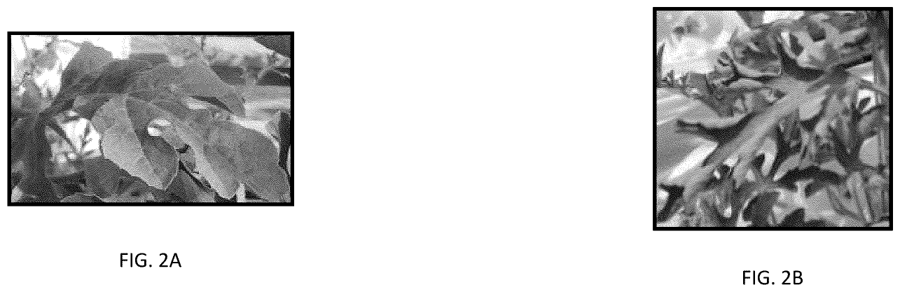

FIG. 3 shows mid-sized seedless watermelon fruit of a drought tolerant plant according to an embodiment of the invention.

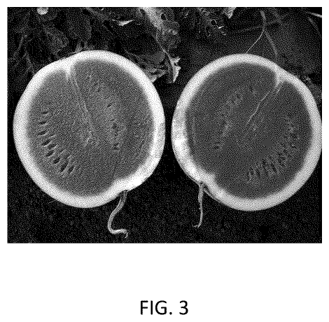

### DETAILED DESCRIPTION

Generally, the present disclosure is based on the unique development of hybrid watermelon plants with agriculturally acceptable tolerance to drought conditions and capable of producing edible fruits. These hybrid watermelon plants are the subject of the first aspect of the present disclosure.

As appreciated, water deficits are one of the most important environmental factors restricting plant growth and productivity, and the genetic improvement of (abiotic and/or biotic) stress tolerance in plants is an urgent challenge for the future of agriculture.

The present disclosure is based on continuous drought resistance applicative trials to establish stable watermelon lines that are tolerant to drought.

In the context of the present invention, when referring to “drought”, “drought stress”, “water stress”, or “drought conditions” (“dry conditions”) it is to be understood as referring to water feed (e.g. water supply by irrigation) of less than 350 M³ per one dunam (1000 m² or 0.1 hectare) containing 500 watermelon plants (or 700 liters per single plant) during a growing period from planting in the open field to reaching mature stage, i.e., ready to harvest, fruit producing plant. This is compared to commonly acceptable water irrigation of watermelon plants in an open field of between 350-450 M³ per one dunam (0.1 hectare or 1000 m²) (or 700-900 liters per single plant). In other words, watermelon plants that are sensitive to water deficiencies, would not reach that mature, edible fruit producing stage, fruit size, as defined herein and production per ton.

A watermelon plant that is agriculturally acceptable tolerant to drought conditions can be defined by the plant's adequate maintenance of its normal functions.

Water is necessary for plants but only a small amount of water taken up, by the roots, is used for growth and metabolism. The remaining 97-99.5% is lost by transpiration (Sinha, Rajiv Kumar (2004-01-01). Modem Plant Physiology. CRC Press. ISBN 978-0-8493-1714-9). Leaf surfaces are dotted with pores called stomata (singular “stoma”), and in most plants they are more numerous on the undersides of the foliage. The stomata are bordered by guard cells and their stomatal accessory cells (together known as stomatal complex) that open and close the pore.

The term “Stoma” or “stomate” as used herein shall include but will not be limited to a pore, found in the epidermis of leaves, stems, and other organs that controls the rate of gas exchange, a pore bordered by a pair of specialized parenchyma cells known as guard cells that are responsible for regulating the size of the stomatal opening, the entire stomatal complex, consisting of the paired guard cells and the pore itself, a stomatal aperture, an aperture through which air enters the plant by gaseous diffusion and contains carbon dioxide which is used in photosynthesis and oxygen which is used in respiration, a plant aperture for diffusing out to the atmosphere Oxygen produced as a by-product of photosynthesis, a plant aperture, for diffusing water vapor into the atmosphere, a pore present in the sporophyte generation of land plant groups except liverworts, and a pore with a size variance across plant species, with end-to-end lengths ranging from 10 to 80 μm and width ranging from a few to 50 μm. Leaves with stomata on both the upper and lower leaf are called amphistomatous leaves; leaves with stomata only on the lower surface are hypostomatous, and leaves with stomata only on the upper surface are epistomatous or hyperstomatous.

The term “Roots” as used herein shall include, but will not be limited to, root structure, root volume, root surface area, root diameter, root length, root to shoot ratio, root development (Waizel et al., 1996).

The term “Turgor pressure” as used herein, shall include but will not be limited to a pressure within cells, regulated by osmosis, a cell pressure causing cell walls to expand during growth, a pressure within cells characterizing the size and rigidity of the cell, a lower Turgor pressure resulting in a wilted cell or plant structure (i.e. leaf, stalk), a cell pressure regulated by the cell's semipermeable membrane, which only allows some solutes to travel in and out of the cell, which can also maintain a minimum amount of pressure, a cell pressure regulated by the transpiration, which results in water loss and decreases turgidity in cells, a large factor for nutrient transport throughout a plant.

Cells of the same organism can have differing turgor pressures throughout the organism's structure. In higher plants, turgor pressure is responsible for apical growth of organs such as root tips and pollen tubes. Turgidity is observed in a cell where the cell membrane is pushed against the cell wall. In some plants, their cell walls loosen at a quicker rate than water can cross the membrane, which results in a cell with lower turgor pressure.

Turgor pressure within the stomata regulates when the stomata can open and close, which has a play in transpiration rates of the plant. This is also important because this function regulates water loss within the plant. Lower turgor pressure can mean that the cell has a low water concentration and closing the stomata would help to preserve water. High turgor pressure keeps the stomata open for gas exchanges necessary for photosynthesis.

The term “Transpiration” as used herein shall include, but will not be limited to, a process of water movement through a plant and its evaporation from aerial parts, such as leaves, stems and flowers, water movement through at least one stomatal aperture and can be thought of as a necessary “cost” associated with the opening of the stomata to allow the diffusion of carbon dioxide gas from the air for photosynthesis, a methodology of also cooling plants, changing an osmotic pressure of cells, and enabling mass flow of mineral nutrients and water from roots to shoots, a water movement through a plant controlled by at least three major factors influencing the rate of water flow from the soil to the roots including: a hydraulic conductivity of the soil and a magnitude of the pressure gradient through the soil wherein both factors influence the rate of bulk flow of water moving from the roots to the stomatal pores in the leaves via the xylem, plant structure and root structure.

Invariably, mass flow of liquid water from the roots to the leaves is driven in part by capillary action, but primarily driven by water potential differences. If the water potential in the ambient air is lower than the water potential in the leaf airspace of the stomatal pore, water vapor will travel down the gradient and move from the leaf airspace to the atmosphere. This movement lowers the water potential in the leaf airspace and causes evaporation of liquid water from the mesophyll cell walls. This evaporation increases the tension on the water menisci in the cell walls and decreases their radius and as well as the tension that is exerted on the water in the cells.

Due to the cohesive properties of water, the tension travels through the stem xylem where a momentary negative pressure is created and to the leaf cells, as water is pulled up the xylem from the roots. As evaporation occurs at the leaf surface, the properties of adhesion and cohesion work in tandem to pull water molecules from the roots, through xylem tissue, and out of the plant through stomata. In taller plants and trees, the force of gravity can only be overcome by the decrease in hydrostatic (water) pressure in the upper parts of the plants due to the diffusion of water out of stomata into the atmosphere and by the capillarity action. Water is absorbed at the roots by osmosis, and any dissolved mineral nutrients travel with it through the xylem.

The cohesion-tension theory explains how leaves pull water through the xylem. Water molecules stick together, or exhibit cohesion. As a water molecule evaporates from the surface of the leaf, it pulls on the adjacent water molecule, creating a continuous flow of water through the plant.

The term “M” as used herein shall include but will not be limited to a cubic meter of pure water at the temperature of maximum density (3.98 C.°) and standard atmospheric pressure (101.325 kPa) has a mass of 1000 kg, or one tonne. At 0 C°, the freezing point of water, a cubic meter of water has slightly less mass, 999.972 kilograms.

The term “M²” as used herein shall include but will not be limited to a square meter, 0.0001 hectares (ha), 0.000247105381 acres or 10.763911 square feet.

The term “Heavy soil” as used herein, shall include, but will not be limited to, a soil rich in fine clay particles.

The term “Medium soil” as used herein, shall include, but will not be limited to, a soil with an average quantity of fine clay particles.

The term “Light soil” as used herein, shall include, but will not be limited to, a soil with a low quantity of fine clay particles.

The term “TSS” as used herein, shall include but will not be limited to an amount of solids dissolved, an amount of solids dissolved measured according to a brix scale, a percent soluble solids (TSS) in a sample of a plant juice, used as an index/parameter for sugar quantity in the fruit (sweetness). The TSS was measured by ATAGO refractometer (http://www.atago.net/USA/products_hsr.php), Brix 0.) to 33.0%, Automatic Temperature Conpensation, The refractometer designed to measure the refractive index of the solution. The Brix percentage represents the total concentration of total soluble solids (TSS) in the sample. At times, the TSS correlates with the plant's fruit general taste. Taste, while being somewhat a subjective characteristic, defines whether the fruit is tasteful, tasteless, bitter and the like.

The term “Brix” as used herein shall include but will not be limited to measuring the percentage of Total Soluble Solids (TSS) in a given substance (per 100 gram), a method to identify the naturally ripened (on-vine) from artificially/post-harvest ripened fruits and a sugar content of an aqueous solution (Degrees Brix (symbol ° Bx)) wherein one degree Brix is 1 gram of sucrose in 100 grams of solution and represents the strength of the solution as percentage by mass. If the solution contains dissolved solids other than pure sucrose, then the ° Bx only approximates the dissolved solid content. The ° Bx is traditionally used in the wine, sugar, carbonated beverage, fruit juice, maple syrup and honey industries.

Comparable scales for indicating sucrose content are the degree Plato (° P), which is widely used by the brewing industry, and the degree Balling, which is the oldest of the three systems and therefore mostly found in older textbooks, but also still in use in some parts of the world.

By way of an example only, a sucrose solution with an apparent specific gravity (20°/20° C.) of 1.040 would be 9.99325° Bx or 9.99359 P while the representative sugar body, the International Commission for Uniform Methods of Sugar Analysis (ICUMSA), which favors the use of mass fraction, would report the solution strength as 9.99249%. Because the differences between the systems are of little practical significance (the differences are less than the precision of most common instruments) and wide historical use of the Brix unit, modem instruments calculate mass fraction using ICUMSA official formulas but report the result as ° Bx.

The content (which solids are dissolved) of solids dissolved, is determined by refractive index by way of using a refractometer and is referred to as the degrees Brix.

The term “Flesh color” of a watermelon flesh shall include but will not be limited to a color varying from scarlet red (dark red), coral red (light red), orange, salmon yellow, canary yellow, or white. In the context of the present disclosure, when referring to red it is to be understood to cover also shades or hues of red, including dark red or red like color. The flesh color may be determined using Pantone Color scale and in accordance with some embodiments, the red color is identified by any one of the Pantone color scale including “red” in their name as available on line at www.pantone.com. In some embodiments, the flesh color is red or dark red.

The term “Vigor” as used herein shall include but will not be limited to a health condition, a hardiness indication, a measure of the increase in plant growth or foliage volume through time after planting, a measurement indicator including biomass production, a number of new shoots produced, a number of reproductive shoots produced, a plant height, a plant volume, a plant height, a plant basal diameter, a number of leaves, a number of stems, a number of leaf whorls, a diameter of rosette, and a volume of plant (height x cover).

Although vigor indicators are also strongly influenced by annual weather patterns, they may be appropriate for some monitoring questions. As nondestructive measures of vigor, they are appropriate for use during the plant growth cycle. Most are easy to measure, with little observer bias.

Vigor characteristics of watermelon plants may be defined by their overall condition or their “vigor” or “strength” as commonly determined in botany (e.g. a measure of an increase in plant growth or foliage volume through time after planting), the vigor being scaled between 10 to 90 on a vigor scale; 10 defining a weak vigor/weak plant and score of “90” on the vigor scale, defining a very strong vigor. Vigor is determined based on plant size and leaves blade size. In some embodiments, vigor of the tolerant plants is between 50-90 on the vigor scale; while vigor of sensitive plant decrease to 10-45 on the vigor scale.

The term “Flesh firmness” as used herein shall include but will not be limited to a force necessary to break the flesh tissue at ripening. Flesh firmness of the watermelon plants disclosed herein was measured by PENETROMETER FRUIT PRESSURE TESTER mod. FT 011 (0-11 lbs.), IRC using an 11 mm plunger attachment. Flesh firmness measures the pressure necessary to force a plunger of specified size into the pulp of the fruit. Such pressure is measured in either pounds or Kilograms. The scale in accordance with the present disclosure, using an 11 mm plunger attachment, was ranked between 1 to 11 lbs., where 1 lbs. indicates that the flesh is very soft, and 11 lbs. indicates a ultra-firm flesh. 1-2 lbs. indicates soft flesh. 2-3 lbs. indicates regular/standard firm flesh. 3-4 lbs. indicates firm flesh. 5-6 lbs. indicates very firm flesh. 7-11 lbs. indicates ultra-firm flesh. Thus, in some embodiments, the flesh firmness of the drought tolerant watermelon fruit is between about 2.5-3.5.

The term “Fruit shape” as used herein shall include but will not be limited to a flat, round, oval and elongated shape. The shape may be defined by the ratio between the major and minor axes of the fruit, such that an oval shape is defined by a ratio between the major axis 1.2-1.4 to the minor axis 1 as a ratio between 1.2:1 to 1.4:1 while in an elongated fruit the ratio is define between 1.5:1 and 1.8:1. Equally, the shape may be scaled between 10 to 90, with 10 representing a flat fruit, 50 round fruit, 60-70 represents an oval fruit and 75-90 represents an elongated fruit. This scale is calculated based on the fruit's measurements ratio (length/width)×50 (a round fruit). Thus, in some embodiments, the fruit shape of the watermelon fruits disclosed herein is between 50 to 70, e.g. ranging from round fruit to oval fruit.

The term “Small fruit size” as used herein shall include but will not be limited to a fruit are typically weighting less than 4.0 kg, at times, between 1.5 and 4.0 kg and at times, between 2 Kg-4 Kg, and are recognized as the mini or personal sized watermelons.

The term “Midi fruit size” as used herein shall include but will not be limited to a fruit weighing 4 Kg-7 Kg.

The term “Large fruit size” as used herein shall include but will not be limited to a fruit weight and at least 7 Kg.

The term “Tolerance” or “Resistance” as used herein shall include but will not be limited to a degree of tolerance to drought, a degree of tolerance to drought determined by phenotypic characteristic and scaled from complete tolerance (or resistance) to sensitive to water stress/drought, a tolerance, resistance or a lack of sensitivity according to a scale of 10 to 90, wherein a “10” indicates a high degree of sensitivity (no tolerance/resistance) which may even results in necrosis, and a “90” indicates a high tolerance/resistance. Thus, it is be understood that a sensitive plant has a low tolerance to draught and high tolerance/resistance (lack of sensitivity) is indicative of high tolerance/resistance to drought. Accordingly, a plant ranked between 50-60 is regarded as intermediate tolerance/resistance, a plant ranked 60-80 is regarded tolerant/resistant, and a plant ranked 80-90 is regarded as highly resistant/highly tolerant. In some embodiments, when referring to tolerance, intermediate resistance or resistance, it is to be understood as referring to a plant comprising a resistance locus being linked to a genetic background or genetic determinant obtainable from the genome of a wild type watermelon plant being agriculturally recognized as having resistance to drought (scale of resistance above 70, and preferably above 80). The comparison between the plants to their tolerance to drought were exhibited in an equal conditions, whereas all the plants received the same water amount (average 200 M³ [190-210] per dunam), in the same field.

The term “Complete tolerance” as used herein shall include but will not be limited to a score of a “90” on the tolerance/resistance scale, and a watermelon plant irrigated under drought conditions and having at least 80%, at times, 90% or even, at times, essentially all of their mature vigorous and green leaves of a watermelon plant grown under non-drought conditions.

The term “Moderate tolerance” as used herein shall include but will not be limited to score of “50-60” on the tolerance scale, and a watermelon plant having at least 80%, at times, 90% or even, at times, essentially all (100%) of their leaves start to fold but while retaining a substantially green color, prior to harvesting.

The term “Weak tolerance” as used herein shall include but will not be limited to score of “30-50” on the tolerance scale, and a watermelon plant having at least 60% or 70% or 80% of their leaves folding and wherein the leaves substantially lose their green color, and become partially or entirely brown, prior to harvesting.

The term “Sensitive” as used herein shall include but will not be limited to score of “10-30” on the tolerance scale, and a watermelon plant having at least 80%, at times, 90% or even essentially 100%, i.e. essentially all of the leaves folded and losing vigor and become brown (i.e. vigor scaled from 10-30 on the vigor scale as discussed herein).

In some embodiments, a plant's normal function under drought conditions is determined by when the plant is grown under drought conditions from seedling stage to harvest, namely, a plant through its setting phase and having ripen fruit.

In some other embodiments, a plant's normal function under drought conditions is determined by a fruit weight being in line with the expected fruit weight based on the selection of the parent line. For example, when a parent line produced large fruits, a plant normal function is one where the hybrid produces large fruits. Thus, in some embodiments, when producing large watermelon producing lines, a normal plant function would be determined by a fruit size of at least 7 kg; or when the watermelon is one yielding midi-sized fruits, normal function is determined by a fruit size of up to 7 Kg, or when the watermelon plant is one yielding mini-sized watermelon fruits up to 4 Kg, all as discussed below.

The present disclosure is based on the development of watermelon plants that are able to exhibit normal plant functions as determined by at least one of the above characteristics, at times, a combination of two, three, four or any other number of combinations of characteristics and yield edible fruits, notwithstanding their cultivation under drought conditions.

In some embodiments, a plant's normal function may be determined by the leave's phenotypic performance. These include, for example, observable characteristics such as leaf performance, e.g. leaf fully opened and green as opposed to folded leaf, vigor lose leaf, wilting and dry brown leaf.

Other characteristics for determining a plant's function include fruit yield, fruit weight, flesh color, flesh firmness, fruit shape, total soluble solids (TSS), existence of seeds (e.g. seed-bearing fruit or seedless), Thousand Seed Weight (TSW), each characteristic constituting a separate embodiment in the context of the present invention. Some of these characteristics are further discussed below.

In some embodiments, at least the leaves' performance (namely, fully opened leaves or folded leaves and leave color being green (normal) vs. yellow or brown (sensitive line) is evaluated in order to scale the level of tolerance/sensitivity to drought conditions. The determination of these characteristics is typically done after a period of cultivation under water stress condition, the period including at least 14 days within the growing period from planting till harvest in the open field under irrigation quantity of 380-420 liter per non-grafted watermelon plant saving on average about 320-480 liters of water per non-grafted watermelon plant.

In yet some other or additional embodiments, tolerance to drought can be determined by growing period from planting till harvest in the open field under irrigation quantity of 380-420 liter per non-grafted watermelon plant saving on average about 320-480 Liters of water per watermelon plant.

Optionally, the determination of these characteristics is typically done after a period of cultivation under water stress condition, the period including at least 14 days within the growing period from planting till harvest in the open field under irrigation quantity of 760-840 liter per grafted watermelon plant saving on average about 640-960 liters of water per grafted watermelon plant.

In yet some other or additional embodiments, tolerance to drought can be determined by growing period from planting (in contrast with seed sowing) until harvest in the open field under irrigation quantity of 760-840 liter per non-grafted watermelon plant saving on average about 640-960 liters of water per watermelon plant.

Planting watermelons (Citrullus lanatus) after sowing the seeds and germination is preferably performed by creating a distance between each non-grafted plant of substantially 1 meter from each other or 2 M² for each plant.

Preferably, when planting, the distance between each grafted plant is substantially 2 meters from each other or 4 M² for each plant.

Preferably, the planting depth of each plant is substantially the depth required to cover the plants roots with and an additional coverage of 1-3 inches (2.5-7.5 cm) Preferably, the time of planting according to the present invention, is January to June (in the northern hemisphere), and July-December (in the southern hemisphere).

Preferably, the time frame from planting to harvest is substantially 82-90 days.

Preferably, soil used is characterized as a Medium to Heavy soil.

Preferably, the methodology of using the fields used for plating includes a practice of growing a series of different types of crops in the same area across a sequence of growing seasons (“Crop rotation”). Crop rotation reduces reliance on one set of nutrients, pest and weed pressure, and the probability of developing resistant pest and weeds.

Crop rotation reduces the need for synthetic fertilizers and herbicides by better using ecosystem services from a diverse set of crops. Additionally, crop rotations improves soil structure and organic matter, which crop rotation reduces erosion, reduces soil borne diseases and increases farm system resilience.

Soil devoid of Crop rotation occurs by growing the same crop in the same place for multiple years in a row, thereby gradually depleting the soil of certain nutrients and selects for a highly competitive pest and weed community. Without balancing nutrient use and diversifying pest and weed communities, the productivity of monocultures is highly dependent on external inputs.

Commonly, watermelons (Citrullus lanatus) are native to dry areas of tropical and sub-tropical Africa, south of the equator, therefore Citrullus lanatus are very demanding in terms of sunlight and temperature throughout the growing period.

The most common reasons for poor germination are over-watering and low temperatures. Preferably, the minimum temperature for germination is 15° C.

Preferably for non-seedless varieties, the temperature for germination is substantially 24-26° C.

Preferably, for seedless varieties, the temperature for germination is substantially 28-32° C.

Preferably, relative humidity of the air for germination is between 95-99%.

Preferably, relative humidity of the soil for germination of non-seedless varieties is between 95-99%.

Preferably, relative humidity of the soil for germination of seedless varieties is between 85-90%.

Preferably, during the first week in the greenhouse, water only is given to the plants in order to keep the growing medium wet.

Preferably, for seedless varieties, a Pollenizer (from a seeded variety) is be situated proximally for readily achieving an exact fruit set, wherein the pollenizer is selected according to a pollinizer with fruits that can be distinguished from the seedless fruits.

Preferably, a pollenizer cultivar is selected for readily producing at least one male flower within a substantially equivalent time frame that at least one female flower appears on the seedless plant.

Preferably, a ratio of three triploids (seedless) plants to one diploid (seeded) plant is created.

The minimum temperature for growth is about 12° C.-15° C., depending on the cultivar. Optimal temperature for vegetative development is between 15-20° C. at night and 25-35° C. during the day.

Preferably, a fertilization program is used, wherein fertilization per hectare is 120-180 Kg N, 150-200 Kg P2O5 and 150-220 Kg K₂.

Optionally, fertilizer rates for Nitrogen (usually in the form of NO₃⁻ and NH₄⁺), phosphorus (P₂O₅), and potassium (K₂O) ranges from 130 kg/hectare-170 kg/hectare Preferably, for grafted watermelon, fertilization of P₂O₅ and K₂ is significantly less and must be adjusted according to local conditions.

Preferably a non-saline sandy or silt loam, or other well-drained soil that hasn't been in cucurbit cultivation for a minimum of 5 years is selected.

Preferably, variations of the amount of fertilization required for watermelon are set according to a soil test to readily determine fertilizer requirements.

Preferably, P and K and half of the N preplant, and then apply the other half of N in-season.

Preferably, a soil pH of between 6-7 is used.

Preferably, in order to achieve an optimal fruit setting, a watering regime is adopted for plants to reach the pollination period in deficient vigorous a state (by avoiding too much water and fertilizer).

Preferably, for the purpose of enhanced pollination, at least one hive per acre (0.4 hectares) is provided. In order not to interfere with the activity of the bees, spraying should be done only with bee-friendly chemicals. Optimal temperature for pollination and fruit setting is 20-25° C.; temperatures up to 35° C. are acceptable. For best fruit quality, there is an advantage to good exposure to sunlight. Fruits reach full maturity 35-45 days after setting in optimal conditions (15-20° C. at night and 25-35° C. during the day).

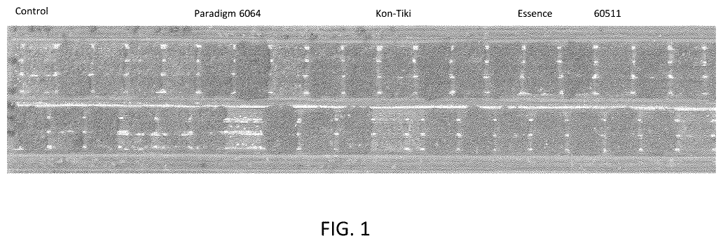

Preferably, and for the purpose of providing a non-limiting example, growing a plurality of Citrullus lanatus varieties including a control group of the Fascination/known-commercial variety is shown in FIG. 1. FIG. 1 shows and demonstrates the outcome of subjecting the progeny watermelon plants to stress conditions comprising water feed of no more than 380-420 liter per plant (in total) until the plant is suitable for harvest (being essentially equivalent to 190-210 M³ per one dunam (0.1 hectare) of 500 watermelon plants.

Preferably, the control group including plants of the Fascination/known-controlled variety is geared towards demonstrating and minimizing the effects of variables other than the selective backcrossing of carefully selected and matched varieties of Citrullus lanatus according to the present invention. Thus, the reliability of the results is increased, by way of an additional comparison between the Fascination/known commercial variety control group measurements and the measurements of the carefully selected and matched varieties of Citrullus lanatus.

Preferably, the selection of the Fascination/known commercial as the control group variety is due to the inherent weak tolerance of the Fascination variety and the weak tolerance of the foliage of the Fascination variety to water scarcity conditions and/or drought condition which are characterized by promptly and substantially loosing Turgor pressure consequently to water scarcity and/or drought conditions.

Accordingly, in some embodiments, tolerance can be scaled as follows:

Thus, in accordance with its first aspect, the present disclosure provides a watermelon plant being characterized by the following features:
it has at least moderate tolerance to drought conditions/water stress;
it produces edible watermelon fruit characterized by at least 10% total soluble solids (TSS) and a red flesh color.

In some embodiments, the TSS of the watermelon fruit is at least 10%, preferably above 12%, or even above 13% or 14%, at times between 13-15%.

In some embodiments, the watermelon plant disclosed herein exhibits a moderate tolerance to drought/dry conditions.

In some embodiments, the watermelon plant disclosed herein exhibits complete tolerance to drought/dry conditions

In the context of the present disclosure, when referring to drought tolerant watermelon, and unless otherwise specified, it is to be understood as encompassing moderate tolerance or complete tolerance, each constituting a separate embodiment of the present disclosure.

A unique feature of the present disclosure is that while the watermelon plant is cultivated under drought condition, i.e. no more than 380-420 liters of water per non-grafted plant from planting until harvesting, it is yet capable of producing edible fruits, at high yield (a fruit setting (the occurrence of fruits setting in a single plant) of at least 1.5 fruits per plant, at times, at least 2.0 fruits per plant, at times between 1.5-3.0 or at times, between 1.5-2.5 fruits per plant) that are red fleshed (red being defined by Pantone color scale), and sweet (TSS of at least 10%, at times in the range of 10% to 14%, at times in the range of 11% to 13.5%). Known-commercial variety performed TSS less than substantially 10% and firmness less than 2 lbs. Origene's tolerant varieties performed TSS higher than substantially 10% and firmness higher than 2 lbs.

In some embodiments of the present disclosure, the fruits are large fruits having a weight larger than 7 kg, such as between 7 kg to 14 kg or 7 kg to 10 kg.

In some other embodiments, the drought tolerant watermelon has mini-sized fruits of between 2-4 Kg.

Irrespective of their size, the watermelon fruits are all sweet, i.e. TSS of at least 10%.

In some embodiments, the tolerant watermelon plant is characterized by seedless fruits. Seedless fruits in the context of the present disclosure denote fruits that lack mature seeds thus lacking the capacity to propagate via seeds. It is to be understood that a seedless fruit may include seed coats and with respect to the present invention a seedless fruit is one comprising between 0-200 seed coats. In some embodiments, the seedless watermelon plant is a triploid hybrid plant.

The watermelon fruits of the drought tolerant watermelon plant disclosed herein can also be characterized by any of the following characteristics, each representing independently an embodiment of the present disclosure:

Deposit—deposit of representative seeds of the lines referred to by the Applicant's internal reference (e.g. Paradigm, ORS 6064 more than 70 seeds per female fruit, ORS 6227 less than 70 seeds per female fruit, Essence and Kon-Tiki more than 70 seeds per female fruit, ORS 60511) and having representative seeds deposited on at the ATCC recognized institute for purposes of patent procedure and according to the present invention,

Preferably, the parental tetraploid plant of a variety selected from the group consisting of Paradigm, ORS 6064, Essence, Kon-Tiki and ORS 60511 have at least 70 seeds per fruit.

In some embodiments, the drought tolerant watermelon plant disclosed herein is characterized by its genetic material, bearing genetic background from a wild type drought resistant watermelon plant known as PI 482312 or from a progeny thereof.

In further embodiments, the drought tolerant watermelon plant disclosed herein is characterized by its genetic material, bearing genetic background from a wild type drought resistant watermelon plant known as Citrullus lanatus var. citroides or from a progeny thereof.

The watermelon plants disclosed herein can be obtained by cultivation using at least one parent with the genetic background of a watermelon having tolerance to drought and under conditions that induce/cause the progeny thereof to be drought tolerant.

Preferably, selection of plants for backcrossing is performed for achieving a greater total root surface area. Due to Nitrogen being mobile within the soil profile and, consequently, bulk flow—is most important for nitrogen uptake (Hill et al., 2006, Bertucci et al., 2018).

Preferably, selection of plants for back crossing is performed according to a root system with longer roots that has a greater total surface area could allow for improved interception of the nitrogen-containing soil solution. As such, those such backcrossing results in an increased surface area and consequent interception of soil solution.

Preferably, the watermelon plants disclosed herein can be obtained by cultivation using at least one parent having a root structure attribute selected from the group consisting of root length, dry weight of the root, surface area of the root, root diameter and the number of lateral roots or “branches” a root has per square inch.

Preferably, the watermelon plants disclosed herein can be obtained by cultivation using at least one parent having a large root surface area and a root structure attribute selected from the group consisting of root length, dry weight of the root, root diameter and the number of lateral roots or “branches” a root has per square inch.

Preferably, the watermelon plants disclosed herein can be obtained by cultivation using at least one parent having a root structure attribute selected from the group consisting of root length, dry weight of the root, surface area of the root, root diameter and the number of lateral roots or “branches” a root has per square inch conjunctively with leaves having stomata selected from the group consisting of a plurality of amphistomatous leaves, and a plurality of hypostomatous leaves. Thus, in accordance with a further aspect, the present disclosure provides a method for producing a watermelon plant characterized at least in that:
(1) the watermelon plant has at least moderate tolerance to drought conditions/water stress; and
(2) the watermelon plant produces edible watermelon fruit characterized by at least 10% total soluble solids (TSS) and a red flesh color; and
(3) the method includes:
   (a) Subjecting at least one progeny watermelon plant to stress conditions comprising water feed of no more than 380-420 liter per plant until said plant is suitable for harvest (being essentially equivalent to 190-210 M³ per 1000M² (one dunam or 0.1 hectare) of 500 watermelon plants); and selecting drought tolerant progenies that will be used as a source to parental lines of first parent.
   (b) Performing at least three backcrosses and at least five self-generations (for the parental line in stage a) are performed for the purpose of enhancing and producing a substantially drought resistant plant and edible and qualitative watermelon fruit (BC3F5).
   (c) Selecting drought tolerant progenies that will be used as a source to parental lines of first parent and having a total soluble solid of at least 10%, red fruit flesh color, and thousand seeds weight (TSW) of between 25 g and 90 g.
   (d) Planting a first watermelon parent being drought tolerant homozygous and having a total soluble solid of at least 10%, red fruit flesh color, and thousand seeds weight (TSW) of between 25 g and 90 g.
   (e) Planting a second watermelon parent that can be not drought tolerant nor sensitive and having a total soluble solid of at least 10%, red fruit flesh color, and thousand seeds weight (TSW) of between 25 g and 90 g. (and also have more than 70 seeds per fruit, as the female parent).
   (f) Crossing the female parent and the male parent (stages a to e) to produce seedless watermelon tolerate to drought.
   (g) Collecting seeds of the watermelon hybrid to produce progeny drought tolerate watermelon plants, and
   (h) Subjecting progeny hybrid watermelon plants to stress conditions including water feed of no more than 380-420 liter per plant until the plant is suitable for harvest (being essentially equivalent to 190-210 M³ per 1000M² (one dunam or 0.1 hectare) of 500 watermelon plants) to confirm the drought tolerance and having an edible and qualitative watermelon fruit.

Thus, in accordance with a yet further aspect, the present disclosure provides a method for producing a watermelon plant characterized by a tolerance to water feed conditions of less than 420 liters per watermelon and edible qualitative watermelon fruit, the method including:
(a) Planting a first watermelon parent being drought sensitive and having a red fruit flesh color, and thousand seeds weight (TSW) of between 25 g and 90 g.
(b) Planting a second watermelon cultivar plant.
(c) Subjecting the second watermelon cultivar plant to stress conditions including water feed less than 420 liters per plant until the second cultivar plant is suitable as a drought tolerant cultivar.
(d) Backcrossing the selected second cultivar with a high quality fruit traits cultivar and selecting for further breeding backcross hybrids that are the most drought tolerant and with high quality fruit traits (at least three backcrosses and at least five self-generation [BC3F5]).
(e) Collecting seeds of the second watermelon cultivar plant resistant to drought conditions, edible and having high quality fruit trait.
(f) Crossing the first watermelon with a plant grown from selected seeds of the second watermelon showing resistance to drought and characterized by its genetic material, bearing genetic background from the drought resistant watermelon plant with the first watermelon.
(g) Collecting seeds of the watermelon F1 hybrid to produce progeny watermelon plants.
(h) Subjecting the progeny watermelon plants to stress conditions including water feed less than 420 liters per plant until the plant is suitable for harvest.
(i) For creating hybrid with drought tolerant male: planting a drought sensitive parent watermelon line.
(j) Pollinating the watermelon line with pollen from the backcross hybrid as a parent watermelon line to produce qualitative watermelon seeds.
(k) Selecting the watermelon seeds produced by step (j) so as to plant only seeds that contain in their genome the most drought tolerant characteristics, and
(l) Planting the watermelon seeds to produce a watermelon plant characterized by resistance to drought.

In some embodiments, drought conditions can be provided by an irrigation schedule as defined in Table 1:

TABLE 1
Water feed from planting until harvest in an open field*

Water Feed

| Group | Liter per plant | M³ per dunam* |
|---|---|---|
| Common farm irrigation | 700-900 | 350-450 |
| Drought conditions | 380-420 | 190-210 |

*500 watermelon plants per 1000 M² (1 dunam or 0.1 hectare)

It is noted that the control group represents common farm irrigation, when cultivating drought sensitive watermelon plants. This is the amount of water feed during the period from planting in the open field until harvest that is required in order to obtain a watermelon plant with normal function and edible fruits.

It is further noted that the drought condition group is received with about 65% to 75%, preferably 70% of water feed as compared to the control group and yet provides edible fruits.

In some embodiment, the first parental watermelon line is a tetraploid and the said another parent watermelon line is a diploid and the progeny watermelon is a triploid seedless watermelon plant.

In some embodiments, both parental lines are diploids and the progeny watermelon is a diploid plant providing seed-bearing watermelon fruits.

At least one of the parental lines, e.g. the male or the female parent are tolerant to drought.

In some embodiments, at least one of the parental lines has a genetic background of at least one of PI 482312 or progeny thereof.

### Description of Non-Limiting Examples

PI 482312 is a watermelon (Citrullus lanatus var. Citroides) line known to have tolerance to drought (Zhang et al., 2011). This PI was obtained from the plant genetic resources unit, Griffin, Georgia, originally from Zimbabwe.

PI 482312, is characterized by a large fruit of about 10 kg, having a gray rind with non-continuous (“broken”) green stripes, white to yellow fruit flesh, non-bitter taste, and Total Soluble Solids (TSS) of 6% in average. The seeds color is light green and big (about 8 mm*13 mm). PI 482312 plants are drought tolerant with a mean drought tolerance rating R=0.9 where R=ΣA×B/EB (A=drought tolerance rating class; B=number of plants in each class) (Zhang et al., 2011).

The development of edible watermelons was based on a cultivation program including open field as follow:

### Open Field Stage:

For genotypes screening, some lines and hybrids were planted in open fields and grown under drought conditions of 190-210 cube per dunam (0.1 hectare) (500 plants per dunam). The growing continued under the same drought conditions until fruit harvesting.

The parents producing the seeds tested in the open field stage were either diploids or tetraploids. In order to produce triploid seedless hybrid plants one parent was a diploid and the other a tetraploid.

Preferably, for the purpose of producing a diploid seed-bearing watermelon plant, two diploid parents were employed. The lines were then crossed with a parental line that is known to produce edible fruits having the size that is desired for the hybrid, e.g. large for large hybrids, midi for midi hybrids and mini for mini hybrids.

1. A watermelon plant characterized by a tolerance to water feed conditions of less than 190-210 M³ per 1000 m² and having properties such that seedless fruits are produced with a flesh firmness of at least 2.0 lbs, measured by PENETROMETER FRUIT PRESSURE TESTER mod. FT 011 (0-11 lbs.), IRC using an 11 mm plunger attachment indicative of a firm flesh, and a red fruit flesh.

2. The watermelon of claim 1, wherein said watermelon is non-grafted.

3. The watermelon of claim 1, wherein said watermelon plant is grafted and said watermelon plant is characterized by a tolerance to water feed conditions of less than 380-420 M3 per 1000 m2 and having properties such that seedless fruits are produced with a flesh firmness of between 2.5-3.5 lbs, measured by PENETROMETER FRUIT PRESSURE TESTER mod. FT 011 (0-11 lbs.), IRC using an 11 mm plunger attachment indicative of a firm flesh, and a red fruit flesh.

4. A watermelon plant characterized by a tolerance to water feed conditions of less than 420 liters per said plant and having properties such that seedless fruits are produced with a flesh firmness of between 2.5-3.5 lbs, measured by PENETROMETER FRUIT PRESSURE TESTER mod. FT 011 (0-11 lbs.), IRC using an 11 mm plunger attachment indicative of a firm flesh, and a red fruit flesh.

5. The watermelon of claim 4, wherein said watermelon is characterized by said watermelon's genetic material, bearing genetic background from a wild type drought resistant watermelon plant known as PI 482312 or from a progeny thereof.

6. The watermelon of claim 4, wherein said watermelon is characterized by said watermelon's genetic material, bearing genetic background from a drought resistant watermelon for readily producing a drought resistant tetraploid watermelon.

7. The watermelon of claim 4, wherein said watermelon is characterized by said watermelon's genetic material, bearing genetic background from a drought resistant watermelon for readily producing a drought resistant diploid watermelon.

8. The watermelon of claim 5, wherein said watermelon variety is selected from the group consisting of Paradigm, ORS 6064, Essence (ATCC deposit no. PTA-127595), ORS6227 and Kon-Tiki.

9. The watermelon of claim 8, wherein said watermelon variety is a progeny of a tetraploid watermelon having at least 70 seeds per fruit.

10. The watermelon of claim 4, wherein said watermelon is characterized by said watermelon's genetic material, bearing genetic background from a drought resistant watermelon plant known as OR60511 or from a progeny thereof.

11. The watermelon of claim 4, wherein said watermelon is characterized by its genetic material, bearing genetic background from a wild type drought resistant watermelon plant known as Citrullus lanatus var. citroides or from a progeny thereof.

12. A method for producing a watermelon plant characterized by a tolerance to water feed conditions of less than 420 liters per watermelon, the method comprising:
(a) planting a first watermelon parent being drought sensitive and having a red fruit flesh color, and thousand seeds weight (TSW) of between 25 g and 90 g;
(b) planting a second watermelon cultivar plant;
(c) subjecting said second watermelon cultivar plant to stress conditions including water feed less than 420 liters per plant until said second cultivar plant is suitable as a drought tolerant cultivar, selected and harvested;
(d) collecting seeds of said second watermelon cultivar plant resistant to drought conditions;
(e) crossing said first watermelon with a plant grown from selected seeds of said second watermelon showing resistance to drought and characterized by its genetic material, bearing genetic background from said drought resistant watermelon plant with said first watermelon;
(f) collecting seeds of said watermelon hybrid to produce progeny watermelon plants; and
(g) subjecting said progeny watermelon plants to stress conditions including water feed less than 420 liters per plant until said plant is suitable for harvest.

13. The method for producing a watermelon plant characterized by a tolerance to water feed conditions of less than 420 liters per watermelon plant of claim 12, wherein said second watermelon is a watermelon of a PI 482312 watermelon line, or a progeny thereof.

14. The method for producing a watermelon plant characterized by a tolerance to water feed conditions of less than 420 liters per watermelon of claim 12, further comprising the steps of:
(h) backcrossing the selected hybrids with a standard drought sensitive variety and selecting for further breeding backcross hybrids that are the most drought tolerant;
(a) planting a drought sensitive parent tetraploid watermelon line;
(b) pollinating said tetraploid watermelon line with pollen from said backcross hybrid as a parent diploid watermelon line to produce triploid watermelon seeds;
(c) selecting the triploid watermelon seeds produced by step (h) so as to plant only seeds that contain in their genome the most drought tolerant characteristics; and
(d) planting said triploid watermelon seeds to produce a triploid watermelon plant characterized by resistance to drought.

15. The method for producing a watermelon plant characterized by a tolerance to water feed conditions of less than 420 liters per watermelon of claim 12, further comprising the steps of:
(h) backcrossing the selected hybrids with a standard drought sensitive variety and selecting for further breeding backcross hybrids that are the most drought tolerant;
(i) planting a drought sensitive parent diploid watermelon line;
(j) pollinating said dipolid watermelon line with pollen from said backcross hybrid as a parent diploid watermelon line to produce diploid watermelon seeds;
(k) selecting the diploid watermelon seeds produced by step (h) so as to plant only seeds that contain in their genome the most drought tolerant characteristics; and
(l) sowing said diploid watermelon seeds to produce a diploid seed-bearing watermelon plant characterized by resistance to drought.

16. The method for producing a watermelon plant characterized by a tolerance to water feed conditions of less than 420 liters per watermelon of claim 12, wherein said a second watermelon is a wild type drought resistant watermelon plant.

17. The method for producing a watermelon plant characterized by a tolerance to water feed conditions of less than 420 liters per watermelon of claim 12, wherein said wild type drought resistant watermelon plant is a watermelon characterized by its genetic material, bearing genetic background from a wild type drought resistant watermelon plant known as Citrullus lanatus var. Citroides or from a progeny thereof.

18. The method for producing a watermelon plant characterized by a tolerance to water feed conditions of less than 420 liters per watermelon of claim 12, wherein the watering regime is substantially 190-210 M³ per one dunam (0.1 hectare) of 500 watermelon plants.

19. The method for producing a watermelon plant characterized by a tolerance to water feed conditions of less than 420 liters per watermelon of claim 18, wherein said second watermelon is a PI 482312 watermelon line, or a progeny thereof.

20. A method for producing a watermelon plant characterized by a tolerance to water feed conditions of less than 420 liters per watermelon, the method comprising:
(a) planting a first watermelon parent being drought sensitive and having a red fruit flesh color, and a thousand seeds weight (TSW) of between 25 g and 90 g;
(b) planting a second watermelon cultivar plant;
(c) subjecting said second watermelon cultivar plant to at least one stress condition including a water feed less than 420 liters per plant until said second cultivar plant is suitable as a drought tolerant cultivar;
(d) backcrossing the selected said second cultivar with at least one high quality fruit trait cultivar and selecting for further breeding a backcross hybrids most drought tolerant and with a high quality fruit trait (at least three backcrosses and at least five self-generation [BC3F5]);
(e) collecting at least one seed of said second watermelon cultivar plant resistant to drought conditions, edible and having a high quality fruit trait;
(f) crossing said first watermelon with a plant grown from at least one selected seed of the said second watermelon, wherein said second watermelon is characterized by resistance to drought and characterized by a genetic material, bearing genetic background from said drought resistant watermelon plant with said first watermelon;
(g) collecting seeds of the watermelon F1 hybrid to produce at least one progeny watermelon plant;
(h) subjecting said progeny watermelon plant to stress conditions including a water feed less than 420 liters per plant until said plant is suitable for harvest;
(i) pollinating said watermelon line with pollen from said backcross hybrid as a parent watermelon line to produce a qualitative watermelon seeds;
(j) Selecting the watermelon seeds produced by step (i) so as to plant only seeds that contain in their genome the most drought tolerant characteristics; and
(k) Sowing said watermelon seeds to produce a watermelon plant characterized by resistance to drought.

21. A method for producing a watermelon plant characterized at least in that:
(a) the watermelon plant has at least a moderate tolerance to drought conditions/water stress;
(b) the watermelon plant produces an edible watermelon fruit characterized by at least 10% total soluble solids (TSS) and a red flesh color; and
(c) the method includes:
(i) subjecting at least one progeny watermelon plant to a stress conditions comprising a water feed of no more than 380-420 liter per plant until said plant is suitable for harvest (being essentially equivalent to 190-210 M³ per 1000M² (one dunam or 0.1 hectare) of 500 watermelon plants);
(ii) selecting at least one drought tolerant progeny for being used as a source to a parental line of a first parent.
(iii) Performing at least three backcrosses and at least five self-generations (for said parental line in said stage said are performed for the purpose of enhancing and producing a substantially drought resistant plant and edible and qualitative watermelon fruit (BC3F5));
(iv) selecting at least one drought tolerant progeny for being used as a source to parental lines of said first parent and having a total soluble solid of at least 10%, a red fruit flesh color, and a thousand seeds weight (TSW) of between 25 g and 90 g;
(v) planting said first watermelon parent being drought tolerant homozygous and having a total soluble solid of at least 10%, red fruit flesh color, and thousand seeds weight (TSW) of between 25 g and 90 g.
(vi) Planting a second watermelon parent being non-drought tolerant nor sensitive and having a total soluble solid of at least 10%, a red fruit flesh color, and a thousand seeds weight (TSW) of between 25 g and 90 g, and wherein said second watermelon parent has at least 70 seeds per fruit, as a female parent;
(vii) crossing said female parent and a male parent (stages a to dc) to produce a seedless watermelon tolerant to drought;
(viii) collecting seeds of a watermelon hybrid to produce a progeny drought tolerant watermelon plant, and
(ix) subjecting said progeny hybrid watermelon plant to a stress condition including water feed of no more than 380-420 liter per plant until the said progeny hybrid plant is suitable for harvest (being essentially equivalent to 190-210 M³ per 1000M² (one dunam or 0.1 hectare) of 500 watermelon plants) to confirm the drought tolerance and having an edible and qualitative watermelon fruit.

22. A non-grafted watermelon plant characterized by a tolerance to water feed conditions of less than 190-210 M³ per 1000 m² and having properties such that seedless fruits are produced with a flesh firmness of at least 2.0 lbs, measured by PENETROMETER FRUIT PRESSURE TESTER mod. FT 011 (0-11 lbs.), IRC using an 11 mm plunger attachment indicative of a firm flesh, and a red fruit flesh said watermelon is characterized by:
(a) said watermelon's genetic material, bearing genetic background from a wild type drought resistant watermelon plant known as PI 482312 or from a progeny thereof;
(b) said watermelon is selected from the group consisting of Paradigm, ORS 6064, Essence (ATCC deposit no. PTA-127595), ORS6227 and Kon-Tiki;
(c) said watermelon variety is a progeny of a tetraploid watermelon having at least 70 seeds per fruit;
(d) said watermelon plants are obtained by cultivation using at least one parent having a root structure attribute selected from the group consisting of root length, dry weight of the root, surface area of the root, root diameter and the number of lateral roots or “branches” a root has per square inch; and
(e) said watermelon plants are obtained by cultivation using at least one parent having leaves with stomata selected from the group consisting of a plurality of amphistomatous leaves, and a plurality of hypostomatous leaves.

23. A method for producing a watermelon plant characterized by a tolerance to water feed conditions of less than 420 liters per watermelon, the method comprising:
(a) planting a first watermelon parent being drought sensitive and having a red fruit flesh color, and thousand seeds weight (TSW) of between 25 g and 90 g;
(b) planting a second watermelon cultivar plant;
(c) subjecting said second watermelon cultivar plant to stress conditions including water feed less than 420 liters per plant until said second cultivar plant is suitable as a drought tolerant cultivar, selected and harvested;
(d) selecting said second watermelon cultivar according to a root structure attribute selected from the group consisting of root length, dry weight of the root, surface area of the root, root diameter and the number of lateral roots or “branches” a root has per square inch;
(e) collecting seeds of said second watermelon cultivar plant resistant to drought conditions;
(f) crossing said first watermelon with a plant grown from selected seeds of said second watermelon showing resistance to drought and characterized by its genetic material, bearing genetic background from said drought resistant watermelon plant with said first watermelon;
(g) collecting seeds of said watermelon hybrid to produce progeny watermelon plants; and
(h) subjecting said progeny watermelon plants to stress conditions including water feed less than 420 liters per plant until said plant is suitable for harvest.

24. A method for producing a watermelon plant characterized by a tolerance to water feed conditions of less than 420 liters per watermelon, the method comprising:
(a) planting a first watermelon parent being drought sensitive and having a red fruit flesh color, and thousand seeds weight (TSW) of between 25 g and 90 g;
(b) planting a second watermelon cultivar plant;
(c) subjecting said second watermelon cultivar plant to stress conditions including water feed less than 420 liters per plant until said second cultivar plant is suitable as a drought tolerant cultivar, selected and harvested;
(d) selecting said second watermelon cultivar according to a leaf structure attribute selected from the group consisting of a plurality of amphistomatous leaves, and a plurality of hypostomatous leaves;
(e) collecting seeds of said second watermelon cultivar plant resistant to drought conditions;
(f) crossing said first watermelon with a plant grown from selected seeds of said second watermelon showing resistance to drought and characterized by its genetic material, bearing genetic background from said drought resistant watermelon plant with said first watermelon;
(g) collecting seeds of said watermelon hybrid to produce progeny watermelon plants; and
(h) subjecting said progeny watermelon plants to stress conditions including water feed less than 420 liters per plant until said plant is suitable for harvest.
